(19) United States
(12) Patent Application Publication
(10) Pub. No.: US 2018/0220960 A1
(43) Pub. Date: Aug. 9, 2018

(54) SYSTEMS AND METHODS FOR MEASURING CALORIC EXPENDITURE

(71) Applicants: Sheng Yang, San Francisco, CA (US); Yen-Chun Yeh, Menlo Park, CA (US); Dominik J. Schmidt, Los Altos, CA (US)

(72) Inventors: Sheng Yang, San Francisco, CA (US); Yen-Chun Yeh, Menlo Park, CA (US); Dominik J. Schmidt, Los Altos, CA (US)

(21) Appl. No.: 15/426,990

(22) Filed: Feb. 7, 2017

(51) Int. Cl. A61B 5/00; A61B 5/01

(52) U.S. Cl. CPC A61B 5/4866; A61B 5/7278; A61B 5/01; A61B 5/4266

(57) ABSTRACT

Embodiments of the present disclosure take advantage of a modified Predicted Heat Strain Model (PHSM) and heat balance equation to accurately determine a calorie expenditure. In various embodiments this is accomplished by using a novel sensor system to gather sensor data comprising a sweat rate and ambient conditions and applying the sensor data to a modified PHSM that distinguishes between normal cases and extreme cases in which body heat can and cannot be efficiently dissipated through sweating. By using a dual method to calculate metabolic rate based on these conditions, the PHSM allows to accurately determine a calorie expenditure.

### BACKGROUND

### A. Technical Field

The present disclosure relates to diagnostic sensor systems. More particularly, the present disclosure related to systems and methods for monitoring and analyzing calorie expenditure.

### B. Description of the Related Art

Developments in sensor technology and mobile communications coupled with ever-increasing computing power have made it possible and relative convenient to continuously monitor human physiological parameters. The integration of lightweight electronic diagnostic sensors into monitoring devices allows the measurement of numerous bodily conditions, such as vital signs, to provide on-the-spot analysis of vast amounts of data in real-time. Yet, the accuracy and reliability of today's wearable gadgets, in particular calorie expenditure monitors that estimate metabolic rate, leaves much to be desired. Typically, commercially available units use algorithms that rely on a basal metabolic rate and sensor data collected from various sensors, such as pedometers and heart rate monitors. The two main sources of measurement inaccuracy are, first, that the relationship between heart rate and oxygen uptake significantly varies from user to user. Second, a variety of physical and non-physical conditions, including stress, illnesses, dehydration, elevated temperatures, and ambient humidity oftentimes contributes to an increase in heart rate, even if the user's oxygen uptake remains unchanged and the metabolic rate does not change. As a result, estimates of burned calories obtained by existing calorie expenditure monitors remain relatively unsatisfactory and, at the most, suitable for consumer electronics devices. More accurate clinical devices, on the other hand, are relatively expensive and impractical for home use.

What is needed are systems and methods that increase accuracy and reliability of calorie expenditure monitors and that allow users to conveniently measure and analyze clinical-grade metabolic rate data.

### BRIEF DESCRIPTION OF THE DRAWINGS

References will be made to embodiments of the invention, examples of which may be illustrated in the accompanying figures. These figures are intended to be illustrative, not limiting. Although the invention is generally described in the context of these embodiments, it should be understood that it is not intended to limit the scope of the invention to these particular embodiments.

FIG. (“FIG.”) 1 is a flowchart of an illustrative process for determining calorie expenditure according to various environment of the present disclosure.

FIG. 2 illustrates an exemplary method for calculating evaporative heat flow for normal conditions utilizing sensor data, according to various environment of the present disclosure.

FIG. 3 is a flowchart of an exemplary process for determining a critical metabolic rate for non-normal conditions, according to various environment of the present disclosure.

FIG. 4 is a flowchart of an exemplary process for iteratively calculating a metabolic rate for non-normal conditions, according to various environment of the present disclosure.

FIG. 5 is a block diagram of an exemplary sensor system for determining a metabolic rate, according to various embodiments of the present disclosure.

FIG. 6 is a flowchart of a generalized process for determining calorie expenditure according to various environment of the present disclosure.

### DETAILED DESCRIPTION OF THE PREFERRED EMBODIMENTS

In the following description, for purposes of explanation, specific details are set forth in order to provide an understanding of the invention. It will be apparent, however, to one skilled in the art that the invention can be practiced without these details. Furthermore, one skilled in the art will recognize that embodiments of the present invention, described below, may be implemented in a variety of ways, such as a process, an apparatus, a system, a device, or a method on a tangible computer-readable medium.

Components, or modules, shown in diagrams are illustrative of exemplary embodiments of the invention and are meant to avoid obscuring the invention. It shall also be understood that throughout this discussion that components may be described as separate functional units, which may comprise sub-units, but those skilled in the art will recognize that various components, or portions thereof, may be divided into separate components or may be integrated together, including integrated within a single system or component. It should be noted that functions or operations discussed herein may be implemented as components. Components may be implemented in software, hardware, or a combination thereof.

Furthermore, connections between components or systems within the figures are not intended to be limited to direct connections. Rather, data between these components may be modified, re-formatted, or otherwise changed by intermediary components. Also, additional or fewer connections may be used. It shall also be noted that the terms “coupled,” “connected,” or “communicatively coupled” shall be understood to include direct connections, indirect connections through one or more intermediary devices, and wireless connections.

Reference in the specification to “one embodiment,” “preferred embodiment,” “an embodiment,” or “embodiments” means that a particular feature, structure, characteristic, or function described in connection with the embodiment is included in at least one embodiment of the invention and may be in more than one embodiment. Also, the appearances of the above-noted phrases in various places in the specification are not necessarily all referring to the same embodiment or embodiments.

The use of certain terms in various places in the specification is for illustration and should not be construed as limiting. A service, function, or resource is not limited to a single service, function, or resource; usage of these terms may refer to a grouping of related services, functions, or resources, which may be distributed or aggregated. Furthermore, the use of memory, database, information base, data store, tables, hardware, and the like may be used herein to refer to system component or components into which information may be entered or otherwise recorded.

Furthermore, it shall be noted that: (1) certain steps may optionally be performed; (2) steps may not be limited to the specific order set forth herein; (3) certain steps may be performed in different orders; and (4) certain steps may be done concurrently.

In order to explain the various embodiments of the present disclosure, several important factors and considerations that affect to how calorie expenditure is determined are described in sections A through G below.

A. Metabolic rate, M

The metabolic rate is treated by ISO 7933 as a known factor, in order to predict the sweat rate and core body temperature. The determination of metabolic rate is described in ISO 8996. In contrast, embodiments of the present disclosure treat metabolic rate as unknown, while other parameters are considered to be known.

B. Heat Flow by Respiratory Convection and Respiratory Evaporation, Cᵣₑₛ & Eᵣₑₛ

Quantification of heat flow by respiratory convection and respiratory evaporation may be expressed by (2) and (3):

$$\begin{matrix}{C}_{res} = 0.072 {c}_{p} \times V \times \frac{{t}_{ex} - {t}_{a}}{{A}_{Du}} & ( 2 ) \\ {E}_{res} = 0.072 {c}_{e} \times V \times \frac{{W}_{ex} - {W}_{a}}{{A}_{Du}} & ( 3 )\end{matrix}$$

where A_{Du} is the Dubois body surface area, cₚ is the specific heat of dry air at constant pressure, cₑ is the latent heat of vaporization of water, V is the respiratory ventilation rate, tₑₓ and tₐ are expired air temperature and ambient air temperature, and Wₑₓ and Wₐ are the relative humidities of the expired air and ambient air. Sensors may be used to measure tₐ and Wₐ. However, other parameters needed to solve equations (2) and (3) are less easily acquired. Empirical expressions that estimate Cᵣₑₛ and Eᵣₑₛ in terms of metabolic rate may be derived from experimental studies of the relationship between the metabolic rate, ventilation rate, temperature, and humidity. These equations are adopted in ISO 7933 as shown in (4) and (5).

Cᵣₑₛ=0.00152M (28.56+0.885t_{α}+0.64 p_{α}) (4)

Eᵣₑₛ=0.00127M (59.34+0.53t_{α}−11.63 p_{α}) (5)

C. Heat Exchanges through Convection and Radiation, C & R

The heat flow by convection and radiation at the skin surface may be expressed by

C=h_{cdyn}×f_{cl}×(tₛₖ−t_{α}) (6)

And

R=hᵣ×f_{cl}×(tₛₖ−tᵣ) (7)

In these equations, h_{cdyn} and hᵣ are the dynamic convective heat transfer coefficient and radiative heat transfer coefficient, respectively, between the clothing and the atmosphere. These variables quantify the impacts of the clothing characteristics, the movements of the subject, and air movement. The variables tₛₖ, tₐ, tᵣ, and f_{cl} represent the mean skin temperature, air temperature, mean radiant temperature, and clothing area factor. The variable h_{cdyn} may be estimated as the larger of 2.38|tₛₖ−tₐ|^{0.25}, 3.5+5.2 vₐᵣ, and 8.7 vₐᵣ^{0.6} with Vₐᵣ denoting the relative air velocity, while hᵣ may be estimated from

$${h}_{r} = 5.67 \times {10}^{- 8} ɛ \times \frac{{A}_{r}}{{A}_{Du}} \times \frac{{( {t}_{cl} + 273 )}^{4} - {( {t}_{r} + 273 )}^{4}}{{t}_{cl} - {t}_{r}} \qquad ( 8 )$$

Detailed definitions and methods for estimating f_{cl}, T_{cl} and Aᵣ/A_{Du}, the ratio of skin surface involved in heat exchange by radiation, are presented in ISO 7933. It is understood that any number of correction factors may be employed, for example, to account for reflective properties of clothing, e.g., by considering different reflection coefficients for different types of clothing material.

D. Heat Flow by Conduction, K

For common activities, such as running, swimming, and weight lifting, subjects are not in contact with solid objects, which is the major route of heat flow by conduction. Therefore, heat flow by conduction may be neglected in the evaluation and set to zero.

E. Effective Mechanical Power, W

Effective mechanical power is the energy spent in overcoming external mechanical forces on the body. As assumed in ISO 7933, in most industrial situations, for which the Predicted Heat Strain Model (PHSM) is designed to evaluate thermal safety, that W is relatively small and may be neglected. In embodiments of the present disclosure, a similar assumption that W may be neglected for normal physical activities, which do not involve storage of extra heat within the body, may be used.

F. Heat Storage, S

When a subject engages in certain activity at the metabolic rate M, the body will approach an equilibrium state with the core temperature determined by M. The relationship between the equilibrium core temperature and metabolic rate may be expressed as

t_{cr,eq}=0.0036(M−55)+36.8 (9)

However, instead of reaching the equilibrium value instantaneously, t_{cr,eq} increases gradually, with a time constant of approximately 10 minutes. Thus, the core temperature at a certain time may be expressed as

$${t}_{cr , eq ( i )} = {t}_{cr , eq ( i - 1 )} \times exp ( \frac{- t}{10} ) + {t}_{cr , eq} \times ( 1 - exp ( \frac{- t}{10} ) ) \qquad ( 10 )$$

where t is measured in minutes. The increase of the core temperature implies that extra heat is stored within the body, instead of being dissipated through sweating. An expression for the instantaneous rate of heat storage, dS_{eq}, is given by

dS_{eq}=cₛₚ×(t_{cr,eq(i)}−t_{cr,eq(i-1)})×(1−α) (11)

where cₛₚ is the specific heat of the body, and α is the fraction of the body mass at skin temperature. In embodiments, both variables cₛₚ and α may be estimated by empirical expressions.

G. Heat Flow by Evaporation at the Skin Surface, E

All the factors are considered as known conditions in the heat balance equation in ISO 7933. By solving the equation, the amount of heat which needs to be dissipated through evaporation at the skin surface, E_{req}, can be calculated as

E_{req}=M−W−Cᵣₑₛ−Eᵣₑₛ−K−C−R−dS_{eq} (12)

As mentioned, it takes time for the body to react to heat stress, and to changes in the metabolic rate. In a similar manner to modeling core temperature, the sweat rate response may be described by a first-order system with a time constant of 10 minutes. A discrete model of the sweat rate at the i-th minute may be expressed in terms of the sweat rate at the (i-1)th minute, and the sweat rate required to maintain thermodynamic equilibrium as shown in (13).

$${SW}_{{p}_{( i )}} = exp ( \frac{- t}{10} ) \times {SW}_{p ( i - 1 )} + ( 1 - exp ( \frac{- t}{10} ) ) \times {SW}_{req} \qquad ( 13 )$$

It is important to note that the ability to dissipate heat through sweating is finite. The maximum sweat rate is affected by several factors, such as sex, age, level of maximal oxygen uptake, ambient temperature, humidity, work intensity, work type, and work duration. Generally, the maximum sweat rate may be estimated by

SWₘₐₓ=(M−32)×A_{d}_{_{M}} (14)

Because the sweat rate is limited, algorithms to predict the sweat rate, SWₚ, and the predicted evaporative heat flow, Eₚ, should model piecewise functions.

Since heat flow by conduction and effective mechanical power may be neglected, the general heat balance equation may be simplified to

M=Cᵣₑₛ+Eᵣₑₛ+C+R+E_{req}+S (15)

where M is the metabolic rate, Cᵣₑₛ is the respiratory convective heat flow, Eᵣₑₛ is the respiratory evaporative heat flow, C is convective heat flow, R is the heat exchange on the skin through radiation, E_{req} is heat exchange on the skin through evaporation, and S is the heat storage.

It is important to note that:

1) heat exchange through convection and radiation, C and R, are independent of the metabolic rate. The values of C and R may be calculated with information collected from sensors and provided by the subjects;

2) heat flow by respiratory convection and respiratory evaporation, Cᵣₑₛ and Eᵣₑₛ, are proportional to the metabolic rate. Also, the coefficients in the expressions for Cᵣₑₛ and Eᵣₑₛ change with environmental conditions; and

3) heat flow by evaporation at the skin surface, E_{req}, and heat storage, S, are constrained by the physiological structure of the body, and the fact that it takes time to react to heat stress and metabolic changes. Thus, these two parameters are affected by metabolic activities and physiological states in a time-dependent manner.

FIG. 1 is a flowchart of an illustrative process for determining calorie expenditure according to various environment of the present disclosure. Process 100 for determining calorie expenditure begins at step 102 when sensor data is received and used to determine an evaporation rate. Sensors may be any type of environmental and physiological sensors that measure and provide environmental and physiological data, such as wearable ambient temperature, skin temperature, and sweat rate, just to name a few.

At step 104, the evaporation rate, which is determined at step 102, is used to determine a metabolic rate for a “normal” condition or range of operation.

At step 106, a threshold metabolic rate is determined for a non-normal condition or range.

At step 108, it is determined whether metabolic rate for the normal condition exceeds the threshold metabolic rate. If so, then, at step 110, sensor data is used to iteratively calculate a final metabolic rate.

Otherwise, at step 112, the metabolic rate for the normal condition is set to the final metabolic rate.

In contrast, ISO 7933 builds a heat equilibrium model, in which E_{req} can be calculated from the given metabolic rate, and dS_{eq} can be derived from the core temperature response associated with certain metabolic activity. The maximum sweat rate must be taken into consideration, if an accurate prediction is expected. When the required skin wetness, w_{req}, exceeds 1.7, saturated water vapor pressure at the surface of the skin exceeds the water vapor partial pressure, or the calculated SW_{req} is larger than SWₘₐₓ, SW_{req} may be substituted by SWₘₐₓ. When SW_{req}>SWₘₐₓ, and the subject is unable to dissipate all the heat generated by current metabolic activity, excess heat is stored in the body. This state may lead to heat stress, and potentially, heat stroke.

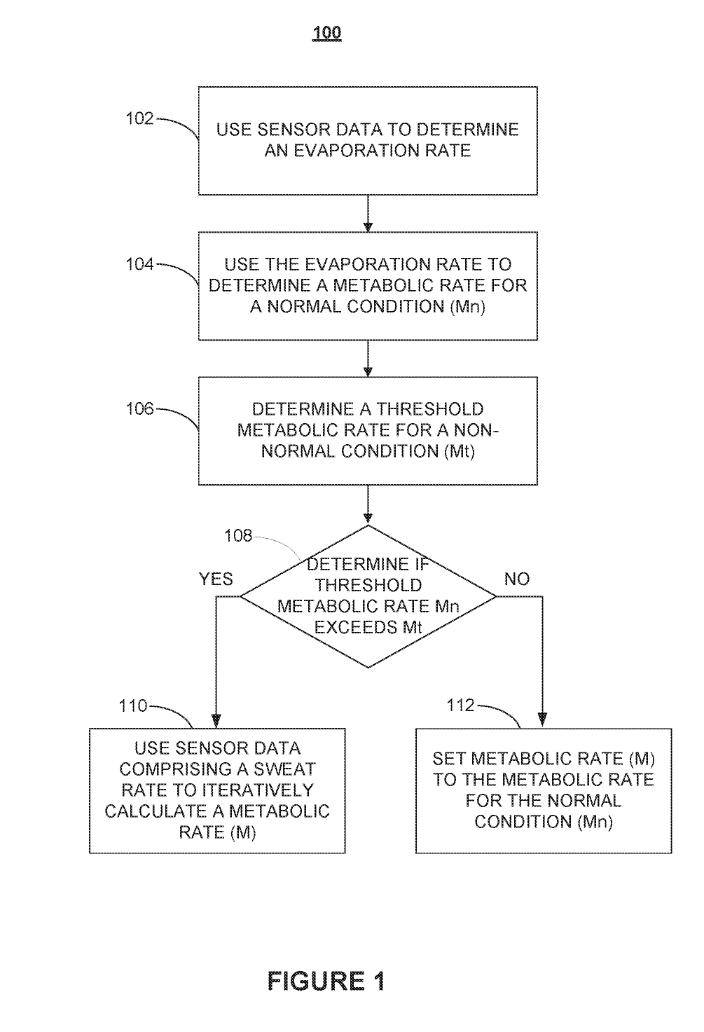

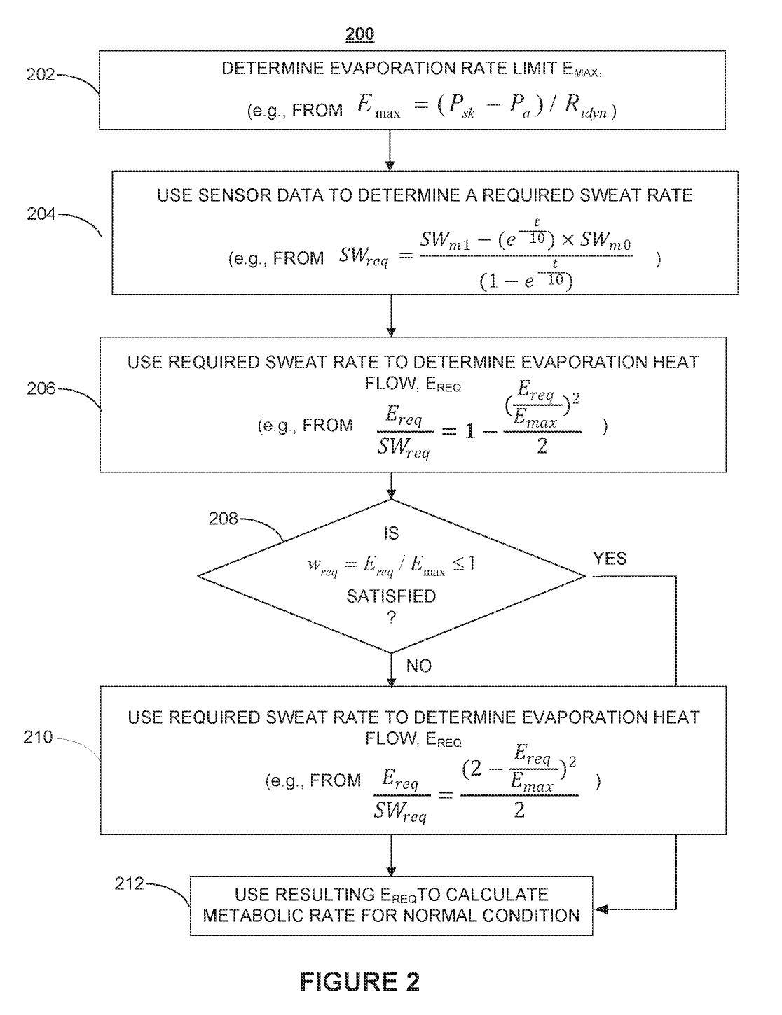

In embodiments of the present disclosure, in scenarios in which SW_{req}≤SWₘₐₓ, a required evaporative heat flow, E_{req}, may be calculated as shown in FIG. 2. FIG. 2 illustrates an exemplary method for calculating evaporative heat flow for normal conditions utilizing sensor data, according to various environment of the present disclosure. Process 200 begins at step 202 when an evaporation rate limit, Eₘₐₓ, is determined from the saturated water vapor pressure at skin temperature, Pₛₖ, which may be calculated from Pₛₖ=0.6105*exp(17.27*Tsk/(Tsk+237.3)), where Tₛₖ is measured using a skin temperature sensor, and Pₐ is the water vapor partial pressure that may be derived from a measurement of humidity, e.g., by a humidity sensor. R_{tdyn} represents the dynamic total evaporative resistance of clothing and boundary air layer.

At step 204, the required sweat rate SW_{REQ} is calculated from a sweat rate, SWₘ₁, at time tⱼ, which may be measured, for example, by a sweat rate sensor, and a sweat rate SWₘ₀ measured at time tᵢ₋₁. In embodiments, the interval between tᵢ−tᵢ₋₁, may be set to, for example, 1 minute.

At step 206, the required sweat rate SW_{req} is used to determine the required evaporative heat flow, E_{req}, for example, from

$$\frac{{E}_{req}}{{SW}_{req}} = 1 - \frac{{( \frac{{E}_{req}}{{E}_{max}} )}^{2}}{2} \qquad ( 16 )$$

by using r_{req}=E_{req}/SW_{req} and the required skin wetness w_{req}=E_{req}/Eₘₐₓ.

If, at step 208, the condition w_{req}=E_{req}/Eₘₐₓ≤1 is satisfied, process 200 may continue with step 212 that uses the resulting E_{req} to calculate the metabolic rate for the normal range, Mn, for the normal range. Otherwise, in embodiments, if the condition is not satisfied, E_{req} may be determined, at step 210, using equation

$$\frac{{E}_{req}}{{SW}_{req}} = 1 - \frac{{( 2 - \frac{{E}_{req}}{{E}_{max}} )}^{2}}{2} \qquad ( 17 )$$

Finally, at step 212, the resulting E_{req} may then be used to calculate the metabolic rate for the normal range. In particular, when the metabolic rate remains within the normal range, dS_{eq} may be determined from E_{req}; and dS_{eq} is linear with respect to the metabolic rate M. Therefore, by replacing the factors in (15) with relevant expressions or values, the heat balance equation (15) may be solved to determine the metabolic rate M.

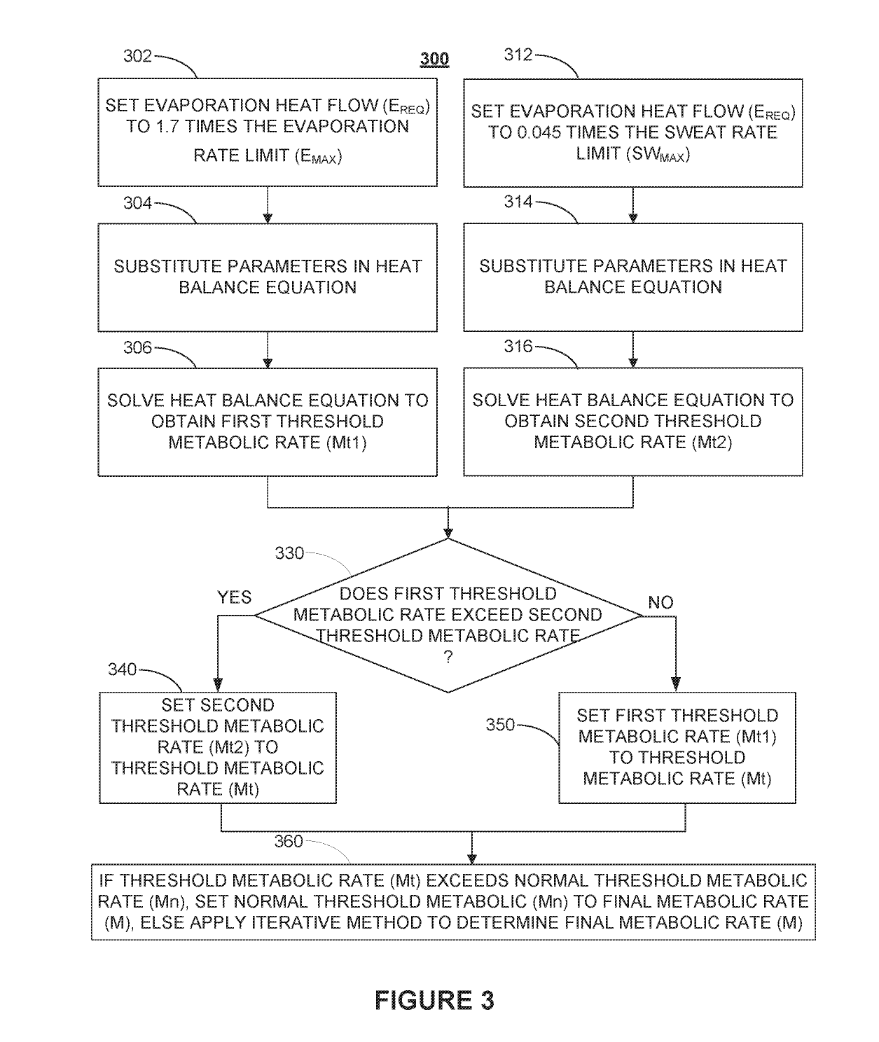

However, this substitution may not be feasible at extreme conditions, such as w_{req}>1.7, or SW_{req}>SWₘₐₓ. Therefore, in embodiments, in order to estimate the heat margin and calculate the metabolic rate M, a critical metabolic rate (or threshold metabolic rate), Mt, is defined. When the subject is working at that critical metabolic rate, conditions are not suitable to dissipate all excess heat through evaporation, and SW_{req} is set to SWₘₐₓ, and the critical metabolic rate may be derived as shown in flowchart in FIG. 3.

FIG. 3 is a flowchart of an exemplary process for determining a critical metabolic rate for non-normal conditions, according to various environment of the present disclosure. Process 300 uses the evaporation rate limit Eₘₐₓ=(Pₛₖ−Pₐ)/R_{tdyn} as calculated in FIG. 2 to determine a first threshold metabolic rate, Mt1, by first setting E_{req} to E_{req}=1.7 Emax at step 302, substituting the resulting value into the heat balance equation at step 304, and solving the heat balance equation M=Cᵣₑₛ+Eᵣₑₛ+C+R+E_{req}+dS_{eq} at step 306. A second threshold metabolic rate, Mt2, may be determined by using equation E_{req}=SWₘₐₓ×r_{req(min)}=0.045×SWₘₐₓ that relates E_{req} to the sweat rate limit SWₘₐₓ in step 312, substituting the relevant parameters into the heat balance equation at step 314, and solving the heat balance equation at step 316.

At step 330, it is determined whether the first threshold metabolic rate, Mt1, exceeds the second threshold metabolic rate Mt2. If so, then, in embodiments, at step 340, the threshold metabolic rate Mt is set to Mt2. Otherwise, at step 350, the threshold metabolic rate, Mt, is set to the second threshold metabolic rate, Mt1.

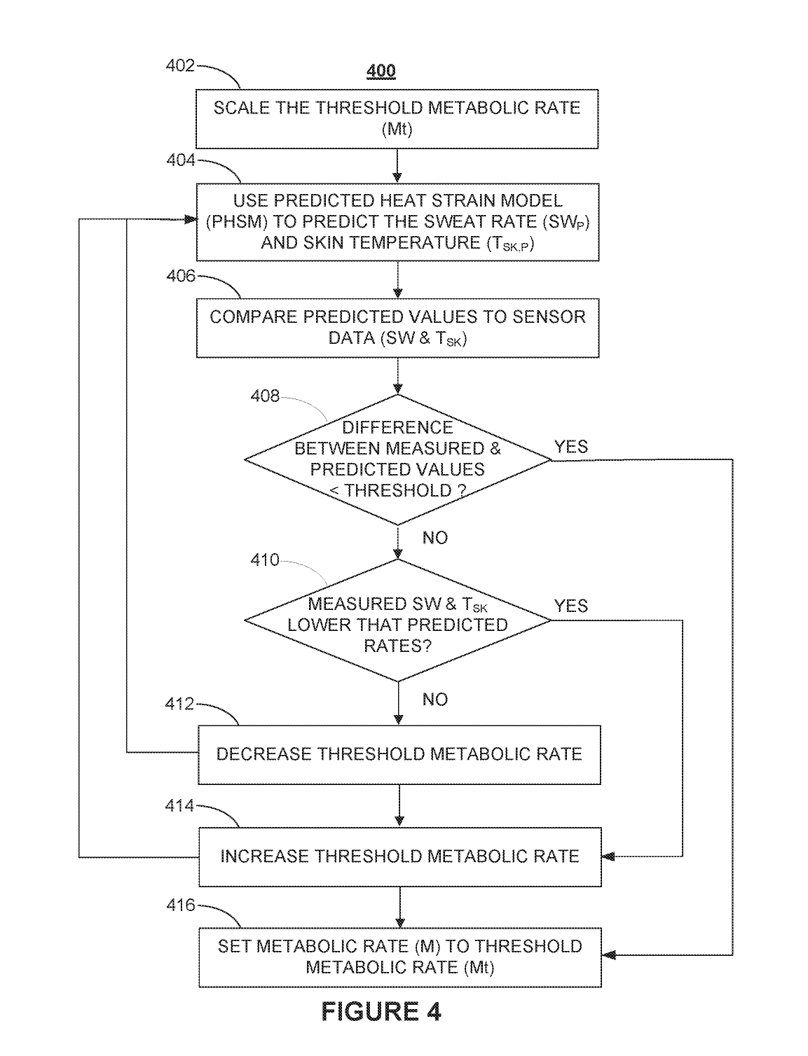

In embodiments, if the threshold metabolic rate is equal to or exceeds the metabolic rate of the normal condition Mn, then Mn is deemed the final metabolic rate. In contrast, when a subject exceeds the threshold metabolic rate, Mt, equation (15) may not be analytically solved. Therefore, in embodiments, an iterative method illustrated in FIG. 4 may be used to estimate the final metabolic rate.

FIG. 4 is a flowchart of an exemplary process for iteratively calculating a metabolic rate for non-normal conditions, according to various environment of the present disclosure. Process 400 for actively calculating the final metabolic rate begins at step 402 when the threshold metabolic rate is scaled to a higher value (e.g., 20%).

At step 404, the PHSM is used to predict the sweat rate and the skin temperature.

At step 406, the predicted values of compared to measured sensor data, and if the difference between the predicted data and the measured sensor data is sufficiently small, e.g., whether |SW-SW_{P}|/(the lesser of SW and SW_{P})<0.01 and |T_{SK}-T_{SKP}|<0.01° C., such that conditions at step 408 are satisfied, the final metabolic rate is set to that threshold metabolic rate. Otherwise, process 400 continues at step 410 when it is determined whether the sweat rate and skin temperature range are lower than their respective predicted rates. If so, at step 414, the critical metabolic rate is scaled to a higher value (e.g., a 5% higher), and process 400 resumes with predicting new sweat rate and skin temperature values, step 404.

Conversely, if, at step 410, it is determined that the sweat rate and skin temperature range are lower than their respective predicted rates, then, at step 412, the critical metabolic rate is scaled to a lower value, and the process resumes with step 404. In embodiments, this recursion is repeated, e.g., until a stop condition is met at which point the final metabolic rate is obtained at step 416. A stop condition may include one or more of the following conditions: a number of predefined iterations have occurred; the predicted and measured values converge to a final value; the predicted and measured values diverge over time; and the difference between successive iterations is reaches an acceptable level of error.

Aspects of the present patent document are directed to information handling systems. For purposes of this disclosure, an information handling system may include any instrumentality or aggregate of instrumentalities operable to compute, calculate, determine, classify, process, transmit, receive, retrieve, originate, route, switch, store, display, communicate, manifest, detect, record, reproduce, handle, or utilize any form of information, intelligence, or data for business, scientific, control, or other purposes. For example, an information handling system may be a personal computer (e.g., desktop or laptop), tablet computer, mobile device (e.g., personal digital assistant (PDA) or smart phone), a network storage device, or any other suitable device and may vary in size, shape, performance, functionality, and price. The information handling system may include random access memory (RAM), one or more processing resources such as a central processing unit (CPU) or hardware or software control logic, ROM, and/or other types of nonvolatile memory. Additional components of the information handling system may include one or more disk drives, one or more network ports for communicating with external devices as well as various input and output (I/O) devices, such as a keyboard, a mouse, touchscreen and/or a video display. The information handling system may also include one or more buses operable to transmit communications between the various hardware components.

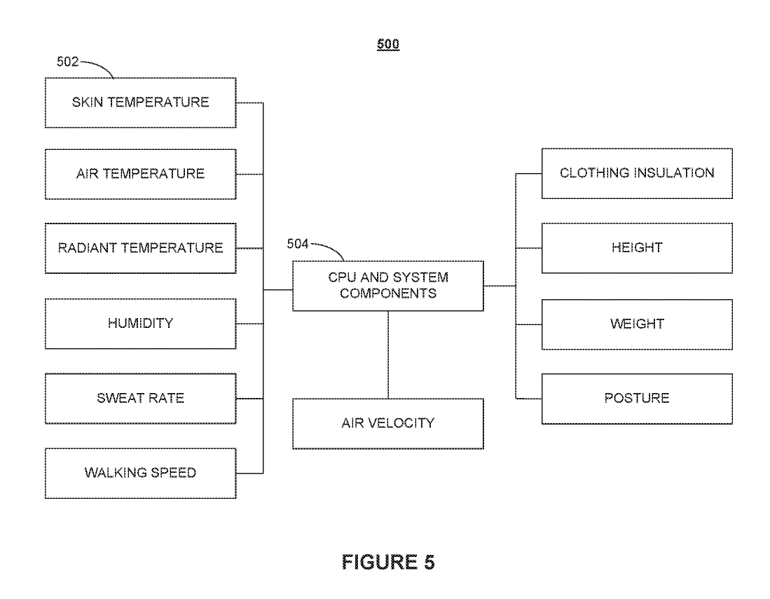

FIG. 5 is a block diagram of an exemplary sensor system for determining a metabolic rate, according to various embodiments of the present disclosure. It will be understood that the functionalities shown for system 500 may operate to support various embodiments of an information handling system—although it shall be understood that an information handling system may be differently configured and include different components. As illustrated in FIG. 5, system 500 includes a CPU 504 that provides computing resources and controls the computer. CPU 504 may be implemented with a microprocessor or the like, and may also include a graphics processor and/or a floating point coprocessor for mathematical computations. System 500 may also include a number of controllers, peripheral devices, and a system memory (not shown not shown in FIG. 5), which may be in the form of random-access memory (RAM) and read-only memory (ROM).

Sensor system 500 may further comprise sensors 502, and a user interface (not shown) to receive user data, such as information related to physical characteristics of the subject for which a metabolic rate is determined.

In embodiments, sensor 502 is a set of sensors designed to monitor physical and physiological parameters comprising skin temperature, air temperature, radiant temperature, water vapor partial pressure, air velocity, walking speed (e.g., via an accelerometer), sweat rate, clothing isolation, weight, height, and posture of the subject. It is understood that one or more parameters may be provided by a single sensor that may have more than one function. Sensor 502 may gather data continuously or at certain periodical of random intervals.

Various sensors may be sensors commonly used in the art or proprietary sensors. For example, temperature sensors, such as the skin temperature sensor or air temperature sensor, may use a thermocouple, a thermistor, or infrared technology. Walking speed may be determined by a pedometer. In embodiments, certain parameters, such as height and weight information, may be entered via a user interface, for example into a mobile device or retrieved from an external or internal database, such as an electronic health care record.

In operation, CPU 504 receives the sensor data (e.g., sweat rate) and other information (ambient conditions) and applies one or more of the methods presented in FIG. 1-4, which may consider normal and extreme cases, to generate an accurate accounting of a calorie expenditure. It is understood that certain parameters may be obtained from additional sources of information or may be provided by a user via the user interface.

In embodiments, system 500 may be coupled to an internal or external database to retrieve additional data or parameters. For example, radiant temperature may be provided by a temperature sensor or may be retrieved from the Internet; similarly, air velocity may be measured or obtained from a weather report. As another example, posture may be determined from video data gathered by a camera that monitors a user's actions. In embodiments, posture is determined by using an accelerometer and applying the gathered data to a machine learning process that analyzes the acceleration and derives posture data therefrom.

In embodiments, clothing insulation is determined in response to a user query that prompts the user to select the type of clothing that the user is wearing, for example, by asking the user to make a choice identifying the type of closing from a number of possible choices from which an evaporative resistance of clothing and/or its boundary air layer may be obtained.

In embodiments, system 500 comprises a storage controller for interfacing with one or more storage devices that include a storage medium, such as magnetic tape or disk, or an optical medium, that may be used to record programs of instructions for operating systems, utilities, and applications, which may include embodiments of programs that implement various aspects of the present invention. Storage device(s) may also be used to store processed data or data to be processed in accordance with the invention. System 500 may also include a display controller for providing an interface to a display device, which may be any type of display. A communications controller may interface with communication devices that enable system 500 to connect to remote devices through any of a variety of networks including the Internet, an Ethernet cloud, a Fiber Channel over Ethernet (FCoE)/Data Center Bridging (DCB) cloud, a local area network (LAN), a wide area network (WAN), a storage area network (SAN) or through any suitable electromagnetic carrier signals including infrared signals.

System components in FIG. 5 may connect to a bus that may represent more than one physical bus. However, various system components may or may not be in physical proximity to one another. For example, input data and/or output data may be remotely transmitted from one physical location to another. In addition, programs that implement various aspects of this invention may be accessed from a remote location (e.g., a server) over a network. Such data and/or programs may be conveyed through any of a variety of machine-readable medium including, but are not limited to: magnetic media such as hard disks, floppy disks, and magnetic tape; optical media such as CD-ROMs and holographic devices; magneto-optical media; and hardware devices that are specially configured to store or to store and execute program code, such as application specific integrated circuits (ASICs), programmable logic devices (PLDs), flash memory devices, and ROM and RAM devices.

Embodiments of the present invention may be encoded upon one or more non-transitory computer-readable media with instructions for one or more processors or processing units to cause steps to be performed. It shall be noted that the one or more non-transitory computer-readable media shall include volatile and non-volatile memory. It shall be noted that alternative implementations are possible, including a hardware implementation or a software/hardware implementation. Hardware-implemented functions may be realized using ASIC(s), programmable arrays, digital signal processing circuitry, or the like. Accordingly, the “means” terms in any claims are intended to cover both software and hardware implementations. Similarly, the term “computer-readable medium or media” as used herein includes software and/or hardware having a program of instructions embodied thereon, or a combination thereof. With these implementation alternatives in mind, it is to be understood that the figures and accompanying description provide the functional information one skilled in the art would require to write program code (i.e., software) and/or to fabricate circuits (i.e., hardware) to perform the processing required.

It shall be noted that embodiments of the present invention may further relate to computer products with a non-transitory, tangible computer-readable medium that have computer code thereon for performing various computer-implemented operations. The media and computer code may be those specially designed and constructed for the purposes of the present invention, or they may be of the kind known or available to those having skill in the relevant arts. Examples of tangible computer-readable media include, but are not limited to: magnetic media such as hard disks, floppy disks, and magnetic tape; optical media such as CD-ROMs and holographic devices; magneto-optical media; and hardware devices that are specially configured to store or to store and execute program code, such as application specific integrated circuits (ASICs), programmable logic devices (PLDs), flash memory devices, and ROM and RAM devices. Examples of computer code include machine code, such as produced by a compiler, and files containing higher level code that are executed by a computer using an interpreter. Embodiments of the present invention may be implemented in whole or in part as machine-executable instructions that may be in program modules that are executed by a processing device. Examples of program modules include libraries, programs, routines, objects, components, and data structures. In distributed computing environments, program modules may be physically located in settings that are local, remote, or both.

One skilled in the art will recognize no computing system or programming language is critical to the practice of the present invention. One skilled in the art will also recognize that a number of the elements described above may be physically and/or functionally separated into sub-modules or combined together.

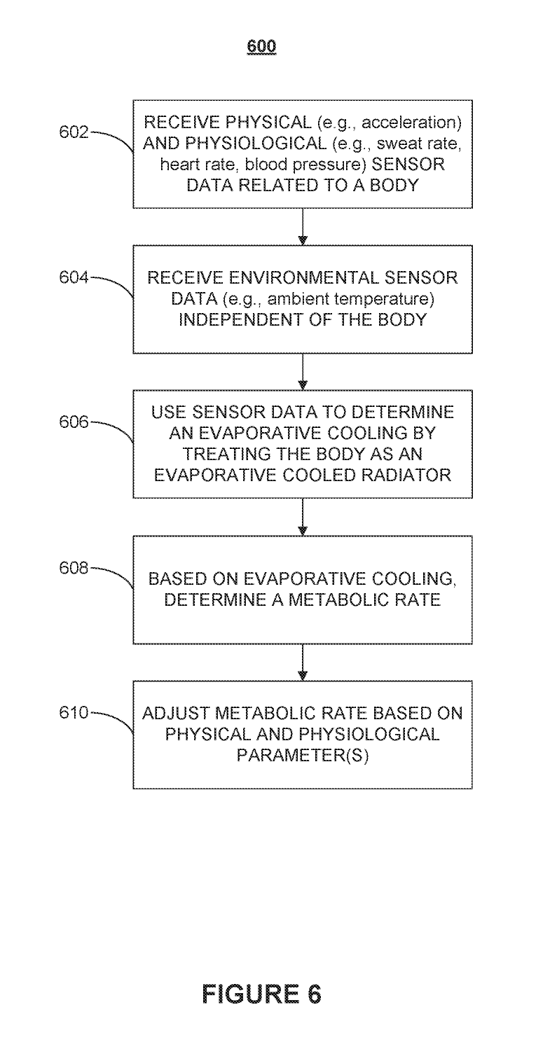

FIG. 6 is a flowchart of a generalized process for determining calorie expenditure according to various environment of the present disclosure. Process 600 begins at step 602 when sensor data is received from a set of sensors. In embodiments, the sensor data comprises physiological parameters that are associated with a body, such as heart rate, blood pressure, or a sweat rate, and physical parameters, such as an acceleration associated with a movement of the body.

At step 604, environmental data, which is independent of the body, is received from one or more environmental sensors, e.g., an ambient temperature sensor.

At step 606, at least some of the sensor data is used to determine evaporative cooling of the body, for example, by treating the body as an evaporative cooled radiator, e.g., a black body radiator, that is subject to at least one of radiation, convection, and conduction related to the body.

At step 608, based on the evaporative cooling, the metabolic rate is determined, for example, by using any of the methods and systems illustrated in FIGS. 1-5.

Finally, at step 610, the metabolic rate is adjusted based on one or more physical and/or physiological parameters to obtain a corrected metabolic rate.

In embodiments, the set of sensors comprises a sweat rate sensor that, in combination with data gathered from environmental sensors, allows to generate accurate metabolic rate data. The sweat rate sensor may dedicated be a dedicated sensor that measure sweat rate or a device that uses skin humidity sensors or measures skin conductivity (e.g., using a galvanic skin response method that measures the electrical conductance of the skin) to determine a sweat rate therefrom.

In embodiments, in cases in which body heat cannot be efficiently dissipated through sweating, an extreme case is defined that is separate and distinct from a normal case. Unlike existing approaches that consider the metabolic rate in the heat balance equation a known parameter and use the heat balance equation in a manner that causes physical and non-physical factors (e.g., heart rate) to falsify readings even if the user's oxygen uptake and metabolic rate remain unchanged, the methods disclosed herein utilize measured sweat rate data to accurately determinate a metabolic rate based on the body's condition. Physiological parameters, in turn, are affected by ambient conditions that are recorded by the environmental sensors.

It shall be noted that elements of the claims, below, may be arranged differently including having multiple dependencies, configurations, and combinations. For example, in embodiments, the subject matter of various claims may be combined with other claims.

It will be appreciated to those skilled in the art that the preceding examples and embodiment are exemplary and not limiting to the scope of the present invention. It is intended that all permutations, enhancements, equivalents, combinations, and improvements thereto that are apparent to those skilled in the art upon a reading of the specification and a study of the drawings are included within the true spirit and scope of the present invention.

What is claimed is:

1. A method for determining a calorie expenditure, the method comprising:
receiving sensor data related to a sweat rate to determine an evaporation rate;
using the evaporation rate to determine a normal metabolic rate for a normal condition;
determining a threshold metabolic rate for a non-normal condition;
determining whether the threshold metabolic rate exceeds the normal metabolic rate; and
in response to the threshold metabolic rate exceeding the normal metabolic rate, iteratively calculating a metabolic rate until a stop condition is met.

2. The method according to claim 1, wherein determining the evaporation rate comprises:
receiving skin temperature data from a skin temperature sensor;
using the skin temperature data to calculate an evaporation rate limit; and
using the sensor data to determine a required sweat rate.

3. The method according to claim 2, wherein the sensor data comprises a physical parameter associated with an environmental sensor that is independent from vital sign data associated with a vital sign sensor.

4. The method according to claim 3, wherein the physical parameter comprises at least one of an acceleration, a humidity, and an ambient temperature.

5. The method according to claim 3, wherein calculating the metabolic rate comprises performing a correction based on at least one of physical characteristics of a person and the physical parameter.

6. The method according to claim 2, wherein the evaporation rate is based on the evaporation rate limit satisfying a first condition.

7. The method according to claim 1, wherein the evaporation rate is based on the required sweat rate exceeding a sweat rate limit.

8. The method according to claim 1, wherein the threshold metabolic rate for the non-normal condition is determined in response to the evaporation rate satisfying a second condition related to a sweat rate limit.

9. The method according to claim 1, wherein iteratively calculating the metabolic rate comprises:
scaling the threshold metabolic rate by a weight factor;
predicting a predicted sweat rate and a skin temperature;
receiving measured sweat rate data and skin temperature data;
comparing the predicted and measured data; and
based on a difference between the predicted and measured data, adjusting the weight factor.

10. The method according to claim 9, wherein the stop condition comprises the predicted and measured data converging to a final value.

11. A system for determining a calorie expenditure, the system comprising:
one or more processors;
sensors designed to monitor physical and physiological parameters to generate sensor data; and
a non-transitory computer-readable medium or media comprising one or more sequences of instructions which, when executed by at least one of the one or more processors, causes steps to be performed comprising:
receiving the sensor data related to a sweat rate to determine an evaporation rate;
using the evaporation rate to determine a normal metabolic rate for a normal condition;
determining a threshold metabolic rate for a non-normal condition;
determining whether the threshold metabolic rate exceeds the normal metabolic rate; and
in response to the threshold metabolic rate exceeding the normal metabolic rate, iteratively calculating a metabolic rate until a stop condition is met.

12. The system according to claim 11, further comprising a user interface to receive user input, and a database that comprises one or more of the physical and physiological parameters.

13. The system according to claim 11, further comprising a camera that monitors a user's actions to determine one or more of the physical and physiological parameters.

14. The system according to claim 11, wherein using sensor data is received from an environmental sensor that measures a physical parameter independent from a sensor that measures vital signs.

15. The system according to claim 14, wherein the physical parameter comprises at least one of an acceleration, a humidity, and an ambient temperature.

16. The system according to claim 11, wherein the one or more processors adjust the sensor data based on at least one of a physical characteristic of a person and a physical parameter.

17. A method for predicting a calorie expenditure, the method comprising:
receiving, from a set of sensors, sensor data that comprises:
a physiological parameter associated with a body;
receiving a physical parameter associated with the body;
receiving an environmental parameter that is independent of the body; and
using at least some of the sensor data to determine an evaporative cooling of the body by treating the body as an evaporative cooled radiator that is subject to at least one of radiation, convection, and conduction related to the body;
based on the evaporative cooling, determining a metabolic rate; and
adjusting the metabolic rate based on at least one of the physical parameter and the physiological parameter.

18. The method according to claim 17, wherein the sensor data comprises a sweat rate that is used to determine an evaporation rate, a normal metabolic rate for a normal condition, and a threshold metabolic rate for a non-normal condition, the method further comprising, in response to the threshold metabolic rate exceeding the normal metabolic rate, iteratively calculating a metabolic rate until a stop condition is met.

19. The method according to claim 18, wherein iteratively calculating the metabolic rate comprises:
assigning a weight factor to the threshold metabolic rate;
predicting a predicted sweat rate and a skin temperature;
receiving measured sweat rate data and skin temperature data;
comparing the predicted and measured data; and
based on a difference between the predicted and measured data, adjusting the weight factor.

20. The method according to claim 17, wherein the set of sensors comprises an environmental sensor and a vital sign sensor that are physically separated from each other by a predetermined distance to reduce an interaction between the environmental sensor and the vital sign sensor.
